(19) United States
(12) Patent Application Publication
(10) Pub. No.: US 2010/0331856 A1
(43) Pub. Date: Dec. 30, 2010

(54) MULTIPLE FLEXIBLE AND STEERABLE ELONGATE INSTRUMENTS FOR MINIMALLY INVASIVE OPERATIONS

(75) Inventors: Christopher R. Carlson, Menlo Park, CA (US); Neal A. Tanner, Mountain View, CA (US); Gregory J. Stahler, San Jose, CA (US); Gene Reis, San Jose, CA (US); Enrique Romo, Dublin, CA (US); Federico Barbagli, San Francisco, CA (US)

Correspondence Address: VISTA IP LAW GROUP LLP, 12930 Saratoga Avenue, Suite D-2, Saratoga, CA, 95070, US

(73) Assignee: HANSEN MEDICAL INC., Mountain View, CA (US)

(21) Appl. No.: 12/637,622

(22) Filed: Dec. 14, 2009

Related U.S. Application Data
(60) Provisional application No. 61/122,298, filed on Dec. 12, 2008.

(51) Int. Cl. A61B 19/00

(52) U.S. Cl. 606/130

(57) ABSTRACT

Configurations are described for conducting minimally invasive medical interventions utilizing elongate robotically controlled instruments and assemblies thereof. In one embodiment, a junction sheath may be utilized to facilitate surgical triangulation of two interventional instrument assemblies, while also directing them to the surgical theater through a minimal single wound or surgical port. One or more instrument drivers, and one or more master input devices comprising an operator workstation, may be utilized to independently and simultaneously control multiple degrees of freedom pertinent to each instrument comprising one or more instrument assembly.

### RELATED APPLICATION DATA

The present application claims the benefit under 35 U.S.C. §119 to U.S. provisional patent application Ser. No. 61/122,298, filed Dec. 12, 2008. The foregoing application is hereby incorporated by reference into the present application in its entirety.

### FIELD OF THE INVENTION

The present invention relates generally to robotically controlled systems, such as robotic or telerobotic surgical systems, and more particularly to flexible and steerable elongate instruments or catheters configured for performing minimally invasive operations.

### BACKGROUND

Standard surgical procedures or open surgeries typically involve using a scalpel to create an opening of sufficient size to allow a surgical team to gain access to an area in the body of a patient for the surgical team to diagnose and treat one or more target sites. When possible, minimally invasive surgical procedures may be used instead of standard surgical procedures to minimize physical trauma to the patient and reduce recovery time for the patient to recuperate from the surgical procedures. However, minimally invasive surgical procedures typically require using extension tools to approach and address the target site, and the typical extension tools may be difficult to use, manipulate, and control. Consequently, only a limited number of surgeons may have the necessary skills to proficiently manipulate and control the extension tools for performing complex minimally invasive surgical procedures. As such, standard surgical procedures or open surgery might be chosen for the patient even though minimally invasive surgical procedures may be more effective and beneficial for treating the patient. Accordingly, there is a need to develop extension tools that are easy to use, manipulate, and control, especially for performing complex minimally invasive surgical procedures.

### SUMMARY

One embodiment is directed to a minimally invasive robotic interventional system, comprising a first instrument driver operatively coupled to a first flexible instrument assembly; a second instrument driver operatively coupled to a second flexible instrument assembly; and an elongate junction sheath having a proximal end, a distal end, and a body portion therebetween, wherein the distal end is coupled to an image capture device and configured to be positioned inside a patient's body adjacent one or more targeted tissue structures, the proximal portion is configured to be positioned external to an opening in the patient's body, and the body portion is configured to contact an entry point of the junction sheath into the patient's body; wherein a distal portion of the of the first flexible instrument assembly is movably coupled through a first lumen defined through the junction sheath between the proximal and distal ends of the junction sheath, wherein a distal portion of the second flexible instrument assembly is movably coupled through a second lumen defined through the junction sheath between the proximal and distal ends of the junction sheath, and wherein the image capture device is positioned substantially equidistantly in between the first and second lumens of the junction sheath to have a field of view configured to capture activity of each of the flexible instrument assembly distal portions. The image capture device may comprise a rod lens, or a chip selected from the group consisting of a CMOS imaging chip and a CCD imaging chip. The image capture device may be movable relative to the elongate junction sheath. The image capture device may be teleoperably moved relative to the elongate junction sheath following commands input by an operator to an operator workstation. The image capture device may be teleoperably moved utilizing an actuation that is electromechanical or manual. The image capture device may be reoriented relative to the elongate junction sheath. The image capture device may be inserted or retracted relative to the elongate junction sheath. At least one of the first and second lumens of the elongate junction sheath may comprise an arcuate lumen configured to cause an instrument coupled therethrough to diverge away from an instrument coupled through the other of the first and second lumens as said instruments exit the distal portion of the elongate junction sheath. Both the first and second lumens of the elongate junction sheath may comprise arcuate lumens configured to cause the first and second instruments to diverge away from each other as they exit the distal portion of the elongate junction sheath. The arcuate lumens may be configured to cause the first and second instruments to converge toward each other as they enter the proximal portion of the elongate junction sheath. The first instrument assembly may comprise an outer sheath instrument coaxially coupled to an inner guide instrument, which is coaxially coupled to a working instrument, and wherein the first instrument driver is configured to independently and simultaneously provide multi-degree-of-freedom actuation to each of the outer sheath instrument, inner guide instrument, and working instrument, in accordance with commands issued by an operator utilizing an operator workstation having a user interface. The user interface may comprise one or more master input devices configured to intake three dimensional positional navigation instructions from the operator pertinent to positional control of at least one point upon one of the instruments comprising the assembly. Two or more master input devices may be utilized to intake three dimensional positional navigation instructions from the operator pertinent to positional control of at least one point upon each of at least two of the instruments comprising the assembly. Each of the outer sheath instrument, inner guide instrument, and working instrument may be remotely controllable in at least six degrees of freedom. The first instrument driver may be configured to pitch, yaw, and insert each of the outer sheath instrument, inner guide instrument, and working instrument independently and simultaneously. The first instrument driver may be configured to roll each of the outer sheath instrument, inner guide instrument, and working instrument simultaneously and together. The system may further comprise a controller configured to automatically reorient the distal portion of the working instrument as the instrument assembly is rolled, to retain positioning of the distal portion of the working instrument while it is being reoriented. The second instrument assembly may comprise an outer sheath instrument coaxially coupled to an inner guide instrument, which is coaxially coupled to a working instrument, and wherein the first instrument driver is configured to independently and simultaneously provide multi-degree-of-freedom actuation to each of the outer sheath instrument, inner guide instrument, and working instrument, in accordance with commands issued by an operator utilizing an operator workstation having a user interface. The system may further comprise a controller configured to coordinate the motion of each of the first and second instrument assemblies relative to each other and relative to the field of view of the image capture device as the instrument assemblies are remotely actuated by independent instrument drivers in accordance with commands from the operator workstation.

### BRIEF DESCRIPTION OF THE DRAWINGS

The present invention will be readily understood by the following detailed description, taken in conjunction with accompanying drawings, illustrating by way of examples the principles of the invention. The objects and elements in the drawings are not necessarily drawn to scale, proportion, precise orientation or positional relationships; instead, emphasis may be focused on illustrating the principles of the invention. The drawings illustrate the design and utility of various embodiments of the present invention, in which like elements are referred to by like reference symbols or numerals. The drawings, however, depict the embodiments of the invention, and should not be taken as limiting its scope. With this understanding, the embodiments of the invention will be described and explained with specificity and detail through the use of the accompanying drawings in which:

FIG. 1 illustrates one embodiment of a robotic or telerobotic surgical system.

FIG. 2A illustrates one embodiment of a multi-armed robotic system.

FIG. 2B illustrates another embodiment of a multi-armed robotic system.

FIG. 3A illustrates another embodiment of a multi-armed robotic system.

FIGS. 3B-3F illustrate aspects of instrument embodiments suitable for use with configurations such as that depicted in FIG. 3A.

FIGS. 4A-4K illustrate cross sectional views of various configurations of main instrument bodies which may be utilized in multi-handed diagnostic and therapeutic interventions.

FIGS. 5A and 5B illustrate an embodiment configured to maintain a targeted object within a field of view of an associated image capture device, and within reach of one or more associated arms.

FIG. 6 illustrates one embodiment of a camera configuration which may be incorporated into a robotic diagnostic or interventional system.

FIGS. 7A-7F illustrate embodiments for incorporating sensory and/or imaging systems into a minimally invasive robotic medical system.

FIG. 8 illustrates another embodiment of a multi-armed robotic system.

FIG. 9 illustrates a further embodiment of a multi-armed robotic system.

FIG. 10A illustrates a mixed sample rate control architecture embodiment without cross coupled devices.

FIG. 10B illustrates a mixed sample rate control architecture embodiment with cross coupled devices.

FIG. 10C illustrates a mixed sample rate “mega bus” control architecture embodiment.

### DETAILED DESCRIPTION

Reference will now be made in detail to the preferred embodiments of the present invention, examples of which are illustrated in the accompanying drawings. While the invention will be described in conjunction with the preferred embodiments, it will be understood that they are not intended to limit the scope of the invention to these embodiments. On the contrary, the invention is intended to cover alternatives, modifications, and equivalents that may be included within the spirit and scope of the invention. Furthermore, in the following detailed description of the present invention, numerous specific details are set forth in to order to provide a thorough understanding of the present invention. However, it will be readily apparent to one of ordinary skilled in the art that the present invention may be practiced without these specific details.

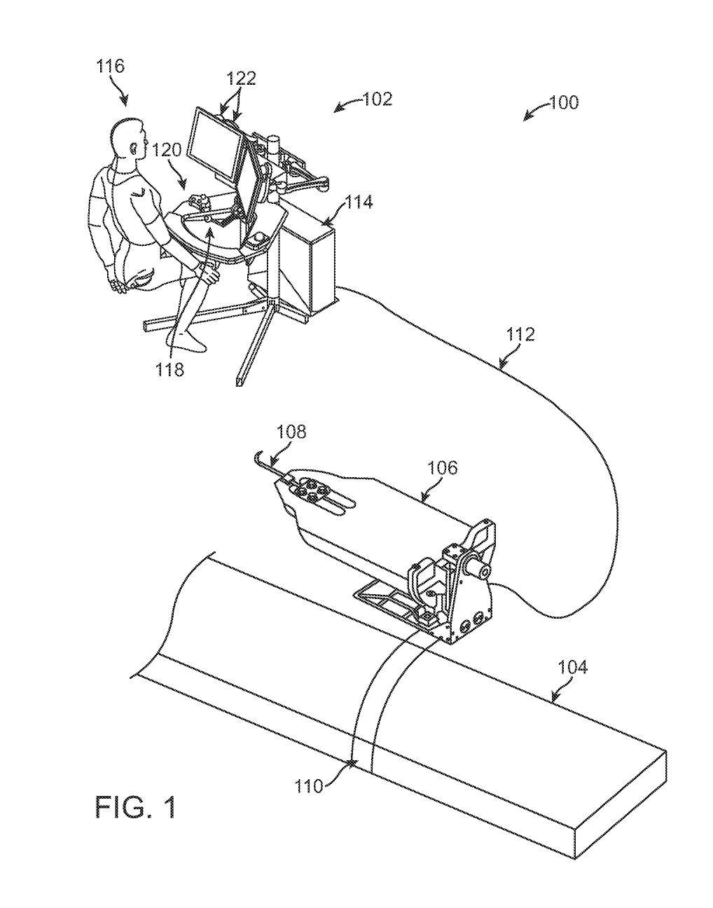

All of the following technologies may be utilized or compatible with manually or robotically steerable instruments, such as those described in the aforementioned U.S. patent application Ser. No. 11/073,363, filed on Mar. 4, 2005; U.S. patent application Ser. No. 11/418,398, filed on May 3, 2006; U.S. patent application Ser. No. 11/637,951, filed on Dec. 11, 2006; U.S. patent application Ser. No. 12/032, 626, filed on Feb. 15, 2008, U.S. patent application Ser. No. 12/032,634, filed on Feb. 15, 2008; U.S. patent application Ser. No. 12/079,500, filed on Mar. 26, 2008; U.S. patent application Ser. No. 12/114,720, filed on May 2, 2008; and U.S. patent application Ser. No. 12/242,196, filed on Sep. 30, 2008. In addition, technologies described herein may be incorporated in conjunction or in combination with technologies described in the aforementioned patent applications. FIG. 1 illustrates one example of a robotic or telerobotic surgical system (100), e.g., the Sensei® Robotic Catheter System from Hansen Medical, Inc. in Mountain View, Calif., U.S.A., with an operator control station (102) located remotely from an operating table (104) to which an electromechanical device, instrument driver, or robotic catheter manipulator (RCM) (106) and instrument assembly or steerable catheter assembly (108), e.g., the Artisan™ Control Catheter also from Hansen Medical, Inc. in Mountain View, Calif., U.S.A., may be supported by an instrument driver mounting brace (110) mounted on the operation table (104). A wired connection (112) transfers signals between an electronics rack (114) at the operator control station (102) and the instrument driver (106) mounted near the operation table (104). The electronics rack (114) includes system hardware and software that operate and perform the many functions of the robotic or telerobotic surgical system (100). The instrument driver mounting brace (110) may be a substantially arcuate-shaped structural member configured to position the instrument driver (106) above a patient (not shown) who is lying on the operating table (104). The wired connection (112) may transmit manipulation, articulation, and control commands from an operator or surgeon (116) who is working at the operator control station (102) and who may be providing the necessary input to the instrument driver (106) by way of one or more input devices, such as an Instinctive Motion™ controller (118), joystick, keyboard (120), trackball, data gloves, exoskeletal gloves, or the like, for operating the instrument assembly (108) to perform various operations, such as minimally invasive procedures, on the patient who is lying on the operating table (104). The wired connection (112) may also transmit information (e.g., visual, tactile, force feedback, position, orientation, shape, localization, electrocardiogram, etc.) from the instrument assembly (108), patient, and operation site monitors (not shown in this figure) to the operator control station (102) for providing the necessary information to the operator or surgeon (116) to facilitate monitoring the instruments, patient, and target site for performing various precise manipulation and control of the instrument assembly (108) during minimally invasive surgical procedures. The wired connection (112) may be a hard wire connection, such as an electrical wire configured to transmit electrical signals (e.g., digital signals, analog signals, etc.), an optical fiber configured to transmit optical signals, a wireless link connection configured to transmit various types of wireless signals (e.g., RF signals, microwave signals, etc.), etc., or any combinations of electrical wire, optical fiber, and/or wireless links. The wire connection (112) allows the surgeon or operator (116) to be remotely located from the patient. The surgeon or operator (116) may be located across the operation room from the patient, in a different room, in a different building, or in a different geographical region away from where the patient is located. Information or feedback transmitted by way of the wire connection (112) may be displayed on one or more monitors (122) at the operator control station (102).

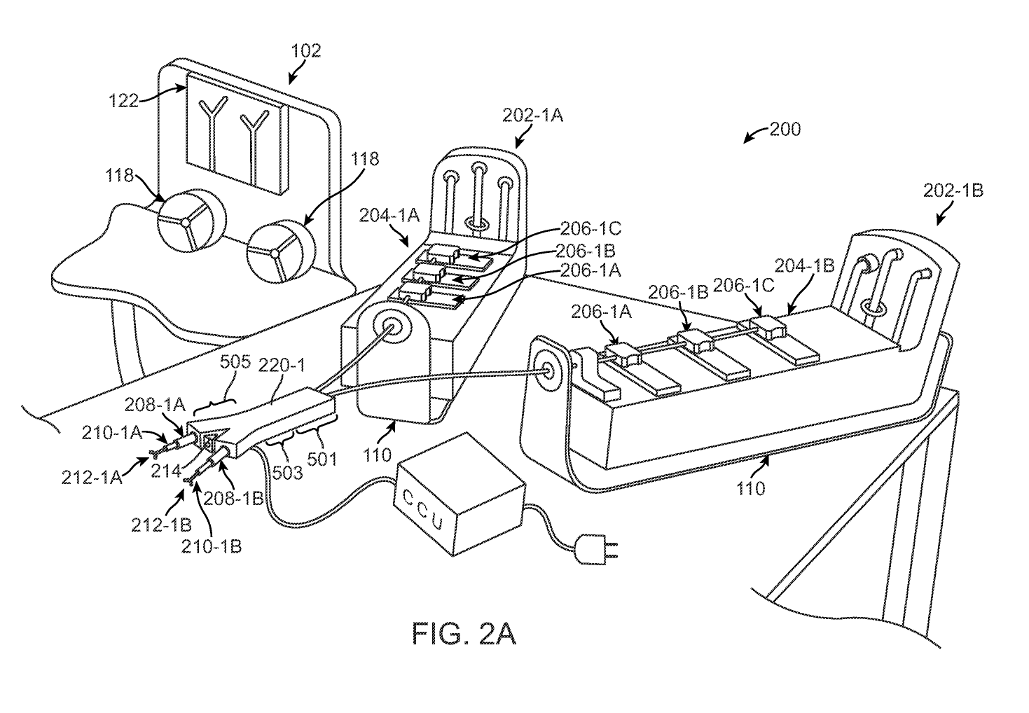

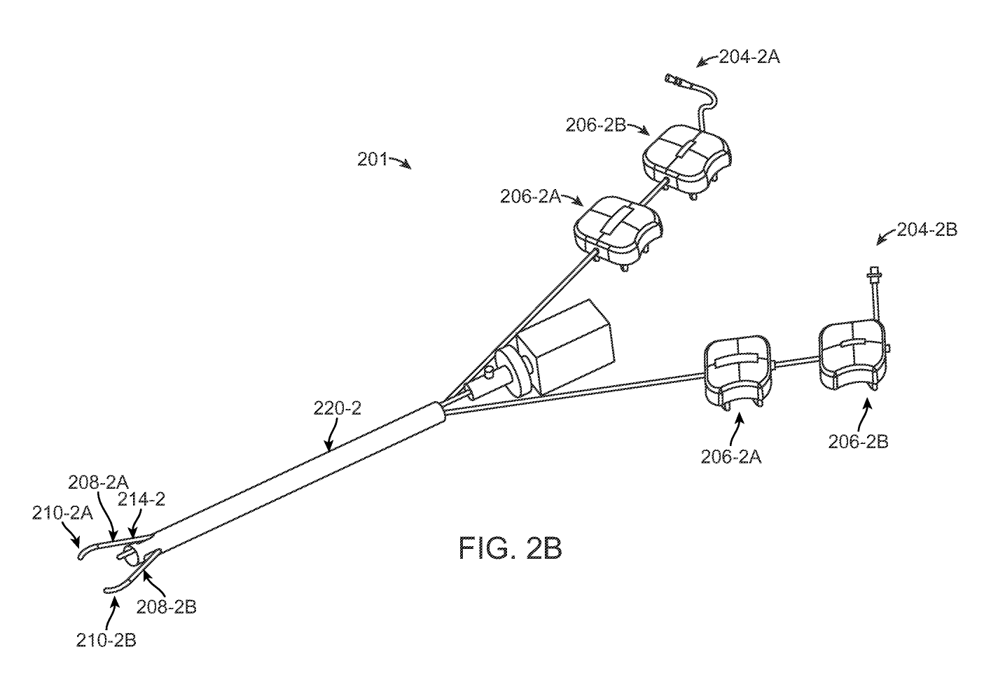

Multiple instrument assemblies may be implemented as a combination to provide a coordinated multi-armed robotic system and approach to assist a surgeon or operator (116) in performing minimally invasive operations. FIG. 2A illustrates one example of a coordinated multi-armed robotic system in which two or more elongate instruments may be operated in concert to perform various minimally invasive diagnostic and/or therapeutic procedures inside a patient. As illustrated in FIG. 2A, this embodiment of a multi-armed robotic system (200) includes two independent instrument drivers (202-1A and 202-1B) and instrument assemblies (204-1A and 204-1B) controlled by one or more instinctive motion controllers, or “master input devices”, (118), which are operatively coupled to a controller, such as a computing system, which preferably is configured to receive commands from the one or more master input devices and other input devices, such as keyboards, control button consoles, and the like, and send motor commands to actuator motors coupled to each of the instrument drivers and configured to actuate the instruments comprising the particular instrument assembly. In other words, the instrument drivers (202-1A and 202-1B) include the mechanisms (e.g., gears, pulleys, belts, motors, etc.) that drive or operate the instrument assemblies (204-1A and 204-1B). The instrument assemblies (204-1A and 204-1B) include splayers or control units (206-1A, 206-1B, and 206-1C), instrument sheaths, or “outer sheath instruments”, (208-1A and 208-1B), instrument guides, or “inner guide instruments”, (210-1A and 210-1B), and working tools, which may comprise end effectors (212-1A and 212-1B). Each of the splayers is configured to mechanically interface with components of the instrument driver, to operate its associated instrument (i.e., instrument sheath (208-1A or 208-1B), instrument guide (210-1A or 210-1B), and working tool/end effectors (212-1A or 212-1B)). For example, the first splayer (206-1A) operates the instrument sheath (208-1A or 208-1B), the second splayer (206-1B) operates the instrument guide (210-1A or 210-1B), and the third splayer (206-1C) operates the instrument tool (212-1A or 212-1B) by means of various control elements, such as gears, pulleys, pull wires, etc., as described, for example, in the aforementioned incorporated by reference documents. In the depicted example, the first splayer (206-1A) may operate the instrument sheath (208-1A or 208-1B) by steering or articulating it in one or more degrees of motions (e.g., up, down, pitch, yaw, etc.) by activating or operating various drive or control mechanisms (e.g., gears, pulleys, pull wires, etc.). The second splayer (206-1B) may operate the instrument guide (210-1A or 210-1B) by steering or articulating it in one or more degrees of motions (e.g., up, down, pitch, yaw, etc.) by activating or operating various drive or control mechanisms (e.g., gears, pulleys, pull wires, etc.). The third splayer (206-1C) may operate the end effectors or instrument tool (212-1A or 212-1B) by activating or operating various drive or control mechanisms (e.g., gears, pulleys, pull wires, etc.). Preferably each of the instruments within an assembly is independently actuated in multiple degrees of freedom, simultaneously. For example, referring to the embodiment of FIG. 2A, each of the depicted instrument assemblies preferably comprises an outer sheath instrument, an inner guide instrument, and a working instrument comprising a distal grasping end effector. Preferably each of the outer sheaths may be inserted/retracted, pitched (positive and negative directions), and yawed (positive and negative directions)—independently and simultaneously. Further, each of the inner guides may be inserted/retracted, pitched (positive and negative directions), and yawed (positive and negative directions)—independently and simultaneously. Further yet, each of the working instruments preferably may be inserted/retracted, pitched (positive and negative directions), and yawed (positive and negative directions)—independently and simultaneously—along with independent and simultaneous grasper actuation for such working instruments. Finally, in the depicted embodiment, each instrument assembly may be rolled together (i.e., each of the instruments in the assembly rolls together positively or negatively). Thus with two independent instrument assemblies, each of the assemblies may be roll actuated independently and simultaneously, but the instruments comprising each of the two assemblies roll together. In one embodiment, the controller is configured to re-orient the distal portion of the working instrument during roll of the associated instrument assembly, to maintain the position of such portion during roll-based reorientation. For example, in a mode wherein maintaining a distal-tip-of-grasper position during roll reorientation is important, such position may be maintained during roll reorientation by commanding the grasper to reorient itself to maintain such position during rolling, preferably through pitch and roll commands causing small reorientations of a distal working instrument wrist or other mechanism configured to allow for grasper orientation adjustment.

Depending on the particular instrument tool that has been implemented onto the instrument assembly (204-1A and/or 204-1B) various motions or articulations may be executed to perform various treatment procedures. For example, as described above, a grasper may be implemented as an instrument tool (212-1A or 212-1B) and the third splayer (206-1C) operate various drive or control mechanisms to control the grasper, such as opening and closing the grasper. In combination with the instrument driver, instrument sheath, instrument guide as well as any distal wrist (such as the wrists available from Novare Surgical, Inc. of Cupertino, Calif.) attachments, the grasper may be advanced, steered, rotated into various positions at a target site to perform various diagnostic or treatment procedures. For instance, one or more graspers may be implemented as instrument tools (212-1A and 212-1B) to perform a suture operation on tissue at a target site inside a patient.

Still referring to FIG. 2A, each of the splayers (206-1A, 206-1B, and 206-1C) and its associated instrument (instrument sheath (208-1A or 208-1B), instrument guide (210-1A or 210-1B), and end effector or instrument tool (212-1A or 212-1B)) may be a substantially modular unit such that the module may be easily separable from the instrument assembly (204-1A or 204-1B). In some embodiments, the splayers (206-1A, 206-1B, and 206-1C) and their respectively associated instruments (instrument sheath (208-1A or 208-1B), instrument guide (210-1A or 210-1B), and end effector or instrument tool (212-1A or 212-1B) may be constructed or fabricated as an integrated assembly and not as separable modular units. Although in this example a two-armed instrument system is shown, other multi-armed robotic system may include more than two arms (e.g., three arms, four arms, five arms, etc.) configured to be operated in concert as a coordinated multi-armed robotic system.

A multi-armed robotic system may include a sensory or imaging system, such as an on-board “image capture device” (214), for observing or monitoring the elongate instruments of an instrument assembly. Suitable image capture devices include CCD imaging chips, CMOS imaging chips, optical fiber bundles, and rod lenses, as described in further detail below. The sensory system or image capture device may be an optical based system, and may be remotely movable or re-orientable utilizing motors, pullwires, flexible and/or steerable sections, and the like. If a multi-armed robotic system is used in a blood field where an optical based system may not be practical, other image capture devices or sensory or imaging systems (e.g., infrared, ultrasound, magnetic resonance imaging, etc.) may be used for observing or monitoring the elongate instruments or arms of an instrument assembly. Referring again to FIG. 2A, an endoscopic system comprising an image capture device (214) may be included in a multi-armed robotic system (200) to provide sensory or image information to observe or monitor the elongate instruments of the instrument assemblies (204-1A and 204-1B) or arms of the robotic system (200). For example, an endoscopic system or image capture device (214) may be incorporated into a main support sleeve or “elongate junction sheath” (220-1). The junction sheath (220-1) may be a substantially rigid structure or it may be a substantially flexible structure. The junction sheath (220-1) may be non-steerable or it may be steerable. The junction sheath (220-1) may be used to support or position the elongate instruments or arms of the robotic system (200). It may also provide a point of reference for maneuvering the arms or elongate instruments. In one embodiment, the junction sheath (220-1) forms two arcuate, or at least partially arcuate, lumens which are configured to accommodate movable coupling with two instrument assemblies, as shown in FIG. 2A, in a manner that allows each of the instrument assemblies to roll and insert relative to the junction sheath (220-1), while also directing the orientations of the instrument assemblies as they enter the proximal portion of the junction sheath, and exit the distal portion. In the embodiment depicted in FIG. 2A, the two arcuate lumens comprising (i.e., formed through or defined by) the proximal portion (501) of the junction sheath (220-1) are configured to bring the instrument assemblies together as they enter the proximal portion (501), while the portions of the two arcuate lumens defined through the distal portion (505) of the junction sheath are configured to cause the two instrument assemblies to divert away from each other as they exit such distal portion (505). This configuration in such embodiment is preferred for surgical triangulation with the distal portions of the instrument assemblies; the distal portions may then be steered back toward each other and utilized to apply capturing and/or compressive loads to a subject tissue structure, etc, with the field of view of the image capture device (214) preferably capturing such activity from a vantage point relatively uncrowded due to the triangulation configuration. The central portion (503) of the junction sheath may be configured to be as small in cross sectional area as possible, and may be positioned approximately at an entry port into the patient from the outside world, to provide a minimally shaped access point; arcuate lumen configurations, as described above, may assist with such functionality, as the lumens are closest together at this middle portion. As described above, the endoscopic system (214) may comprise a fiber optic system, a CCD camera system, a CMOS system, combination of CCD and CMOS, a laser system, infrared system, ultrasound, or other suitable sensory or imaging system.

FIG. 2B illustrates another embodiment of a multi-armed robotic system.

FIG. 2B illustrates a multi-armed robotic system (201) that includes two instrument drivers (not shown) and instrument assemblies (204-2A and 204-2B) controlled by one or more instinctive motion controllers or master input devices (not shown). The instrument drivers include the mechanisms (e.g., gears, pulleys, belts, etc.) that drive or operate the instrument assemblies (204-2A and 204-2B). The instrument assemblies (204-2A and 204-2B) include splayers or control units (206-2A and 206-2B), instrument sheaths (208-2A and 208-2B), instrument guides (210-2A and 210-2B). In this embodiment, end effectors or instrument tools may not be coupled to each of the instrument guides (210-2A and 210-2B) as illustrated in FIG. 2A, instead instrument tools may be passed through each respective lumen of the instrument assembly (204-2A and 204-2B) from the proximal portion of the instrument assemblies to the distal portions at the tips of the instrument guides (210-2A and 210-2B) to perform various operations. Each of the splayers (206-2A and 206-2B) operates its associated instrument (i.e., instrument sheath (208-2A or 208-2B) and instrument guide (210-2A or 210-2B)). For example, the first splayer (206-2A) operates the instrument sheath (208-2A or 208-2B), and the second splayer (206-2B) operates the instrument guide (210-2A or 210-2B) by way of various control elements, such as gears, pulleys, pull wires, etc. In this example, the first splayer (206-2A) may operate the instrument sheath (208-2A or 208-2B) by steering or articulating it in one or more degrees of motions (e.g., up, down, pitch, yaw, etc.) by activating or operating various drive or control mechanisms (e.g., gears, pulleys, pull wires, etc.). The second splayer (206-2B) may operate the instrument guide (210-2A or 210-2B) by steering or articulating it in one or more degrees of motions (e.g., up, down, pitch, yaw, etc.) by activating or operating various drive or control mechanisms (e.g., gears, pulleys, pull wires, etc.). The “arms” of this multi-armed robotic system which may be comprised of instrument sheaths (208-2A and 208-2B) and instrument guides (210-2A and 210-2B), and they may be disposed within a main, or junction, sheath (220-2). In some embodiments, the junction sheath (220-2) may be a substantially rigid elongate instrument, and may be configured to accommodate a substantially rigid instrument portion, such as an elongate rod lens; however, in some other embodiments, the main sheath (220-2) may be a substantially flexible and pliable elongate instrument which may easily conform to the shape of tortuous natural pathways within the body of a patient. In some embodiments the junction sheath (220-2) may not be steerable. It may be configured to passively conform to the shape or curvature of the tortuous natural pathways as it is advanced forward a target site. In some other embodiments, the junction sheath (220-2) may be steerable such that it may be steered or directed down specific branches of tortuous natural pathways in a patient. Referring back to FIG. 2B, the arms of this robotic system (201) may be partially or completely withdrawn into the main sheath (220-2) when the main sheath is being advanced to a target site inside a patient. Once the main sheath (220-2) is advanced to a location near the target site, the flexible and steerable arms may be advanced out of the ports on the main sheath and further steered or maneuvered into position to perform various diagnostic or therapeutic operations. The appropriate surgical instruments may be advanced through each lumen of the instrument assemblies (204-2A and 204-2B) to execute the various diagnostic or therapeutic operations.

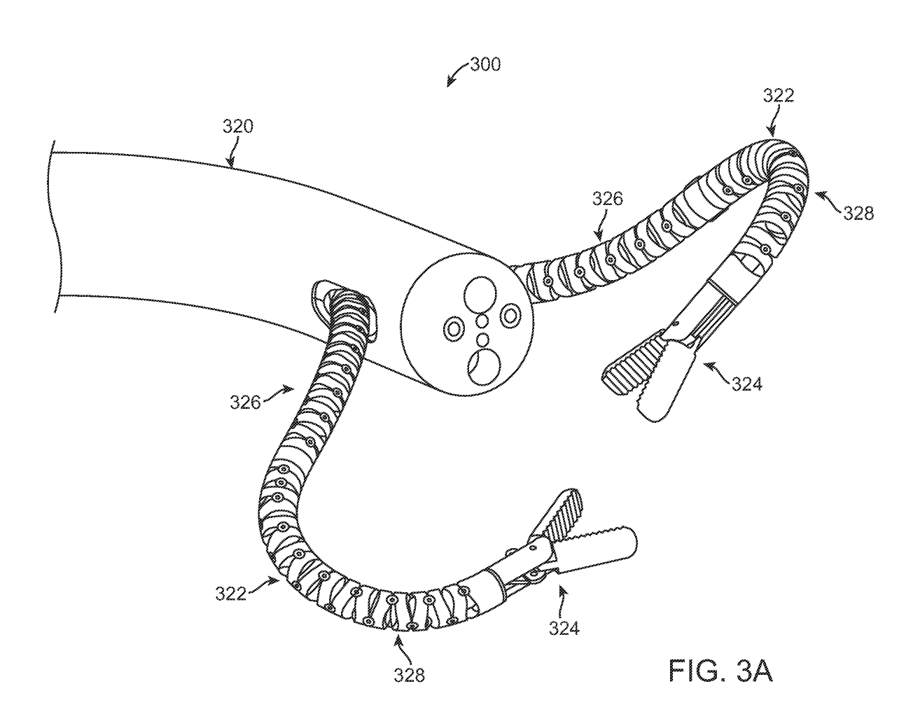

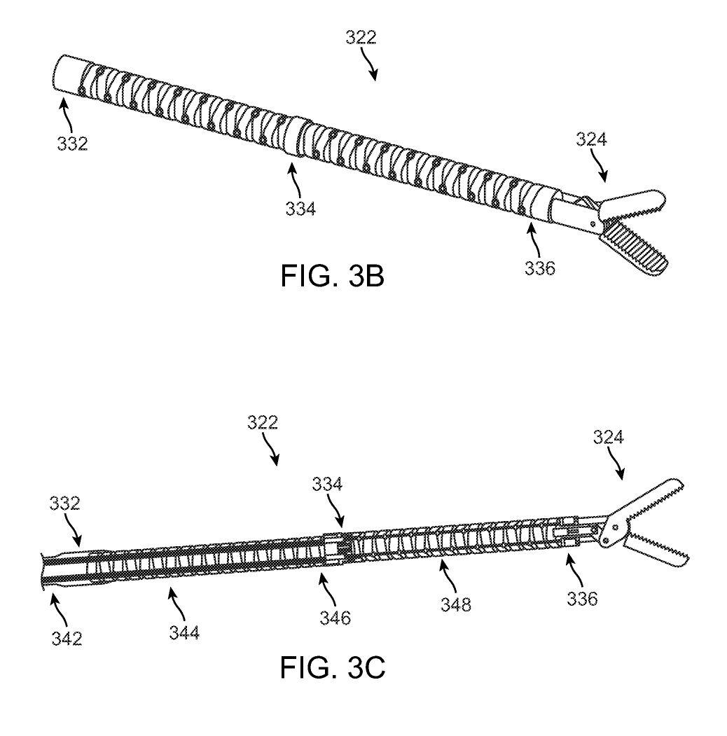

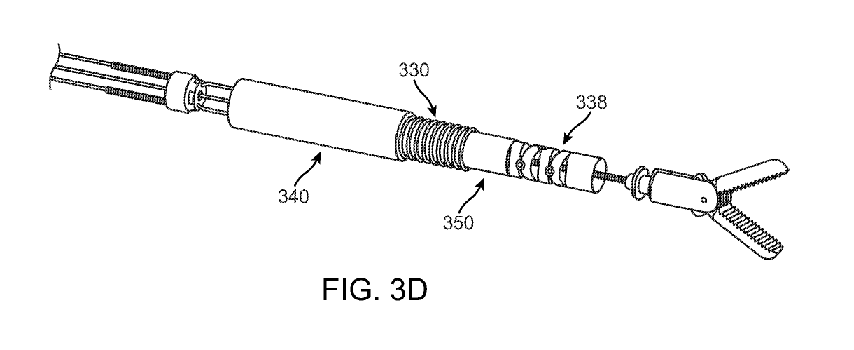

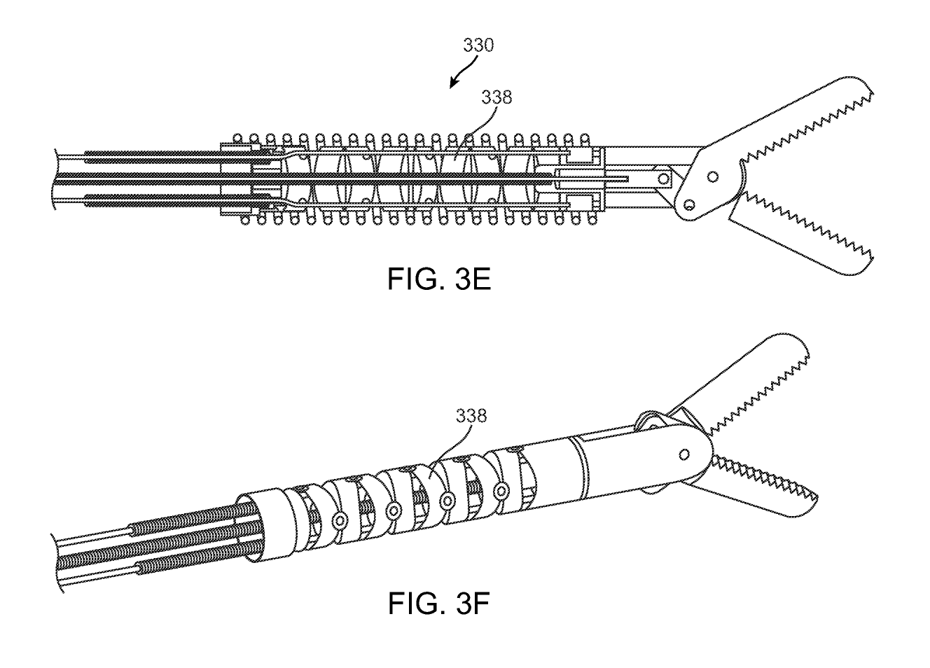

FIG. 3A illustrates another embodiment of a multi-armed robotic system (300). In this embodiment, the robotic system (300) includes integrated flexible and steerable arms (322) and end effectors or instrument tools (324), which may be extended from the ports on the main flexible instrument body. The robotic arms (322) include a proximal segment (326) and a distal segment (328). In addition, the robotic arms include a proximal base (332), a distal base (334), and a distal tip (336), as illustrated in FIG. 3B. The proximal base (332), distal base (334), and distal tip (336) provide support to the control elements of the robotic arm (322) as illustrated in FIG. 3C. The control elements of the robotic arm (322) may include coil pipes (342 and 344) and pull wires (346 and 348). Both the proximal segment (326) and the distal segment (328) may include respective coil pipes (342 and 344) and pull wires (346 and 348). For example, the distal end of one set of coil pipes (342) may terminate and couple to the robotic arm structure at the proximal base (332). This structural configuration allows the proximal segment (326) to be decoupled from the base of the robotic arm. In addition, the distal end of another set of coil pipes (344) and pull wires (346) may terminate and couple to the robotic arm structure at the distal base (334), while another set of pull wires (348) may terminate and couple to the robotic arm structure at the distal tip (336). This structural configuration allows the distal segment (326) to be decoupled from the proximal segment of the robotic arm when the pull wires (348) are operated to steer the distal segment (328) of the robotic arm (322). The articulation force applied to steer the distal segment may have negligible effect on the proximal segment (326). The articulation force may be decoupled from the body or structure of the robotic arm at the distal base (334) where the coil pipe (344) may be coupled to the structure of the robotic arm and through this coupling point, the articulation force may be transmitted to the coil pipe (344). As may be appreciated, the proximal and distal segments (326 and 328) may be steered in one or more degrees freedom or motions (e.g., up, down, pitch, yaw, etc.) as well as roll relative to the main instrument (320). In addition, the robotic arms (322) may be translated in and out of the ports of the main instrument, or junction sheath, (320) to position the end effectors or instrument tools (324). The coil pipes may minimize any impact to the acquired or articulated positions or shapes of the proximal segments (326). The end effectors or instrument tools may include their associated coil pipes and pull wire for articulation, control, and/or operation. FIGS. 3D through FIG. 3F illustrate further details of related configurations. Referring to FIG. 3D, in order to maintain a consistent radius of curvature throughout a pivot spine (338) type bending section when articulated, a coil spring (330) may be added over a portion of, or the entirety of, the segment. The spring provides structural consistency throughout the segment, therefore minimizing any preferred bending positions inherent to the pivot spine design. The spring also increases the overall stiffness, which improves the instrument's ability to maintain an articulated shape when exposed to external forces. A mesh (350) may be added directly over the pivot spine (338) in order to improve column stability by minimizing rotational displacement between the first and last spine (338) links. The mesh (250) preferably will also provide a barrier between the coils on the spring (330) and the links of the pivot spine (338), minimizing the risk of mechanical catching between both components. A jacket (340), such as a polymeric layer or tubing component, may be added over the spring (330) in order to protect the assembly below, to improve aesthetics, and/or to improve reprocessing capabilities to minimizing hard-to-clean features. FIG. 3E depicts a partial cross sectional view showing a pivot spine (338) assembly with a coil spring (330). FIG. 3F depicts an orthogonal view showing the pivot spine (338) in detail.

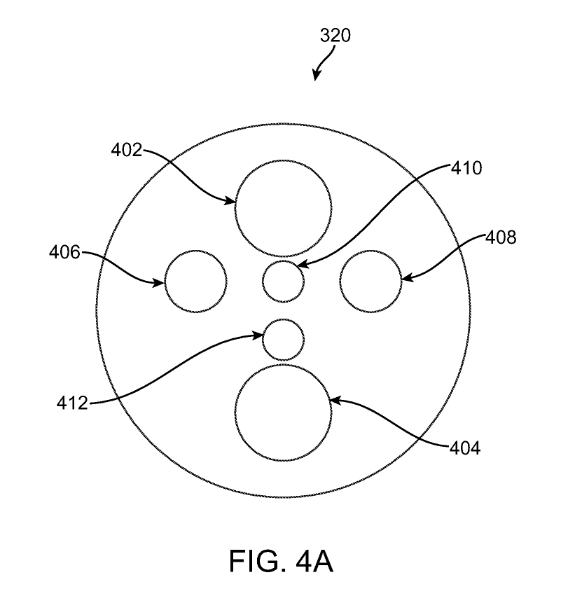

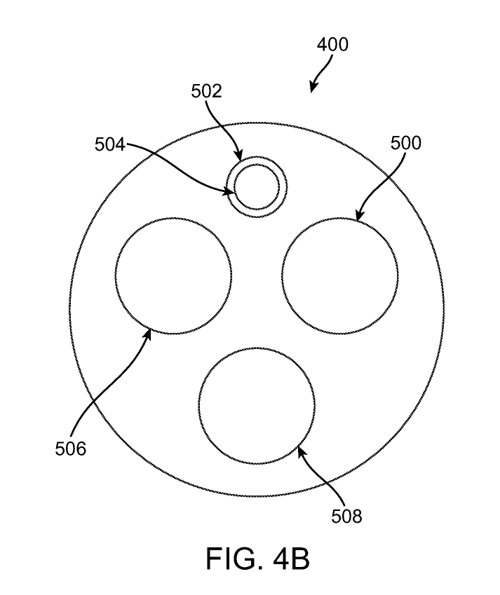

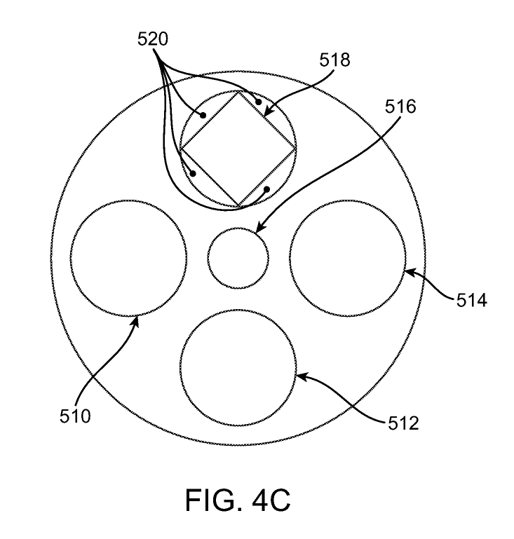

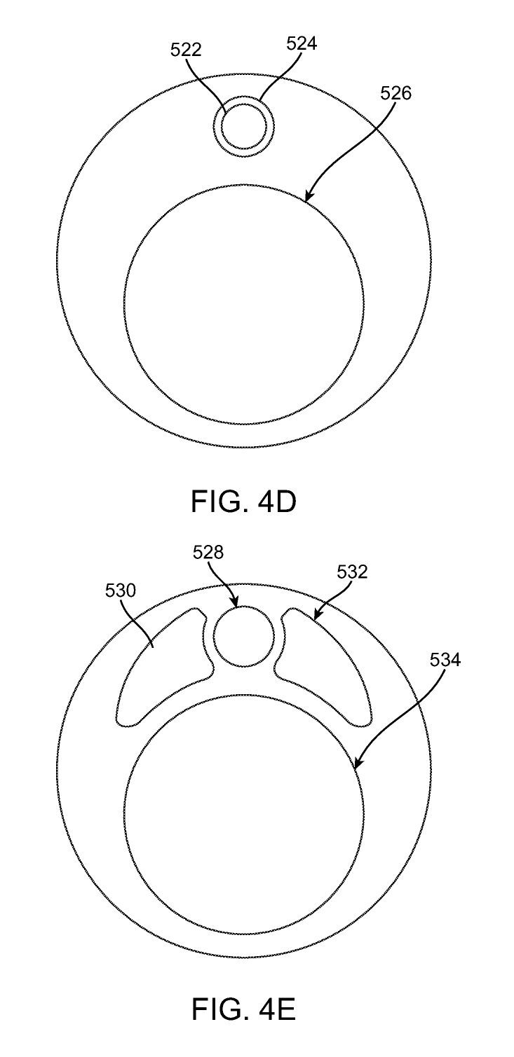

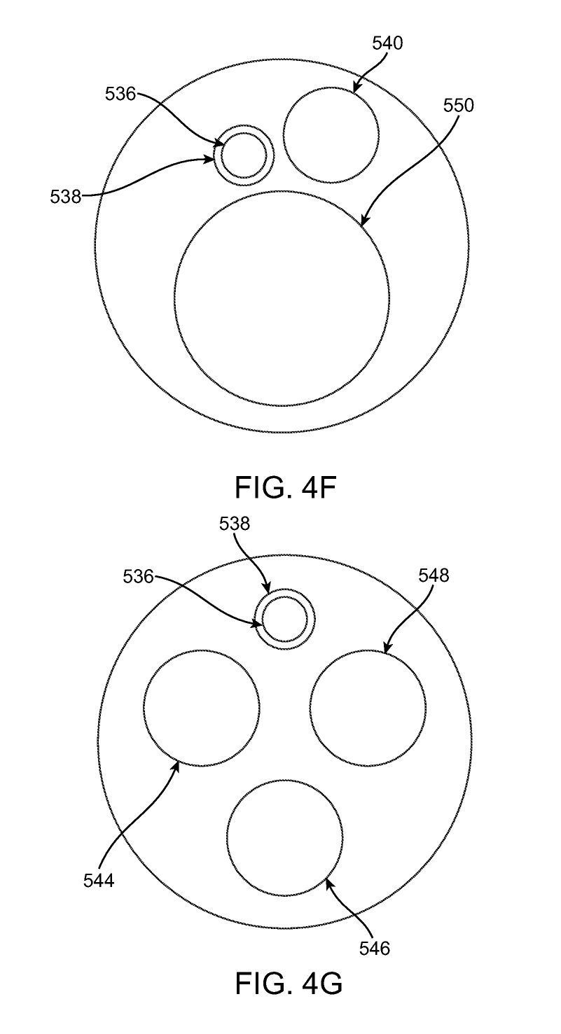

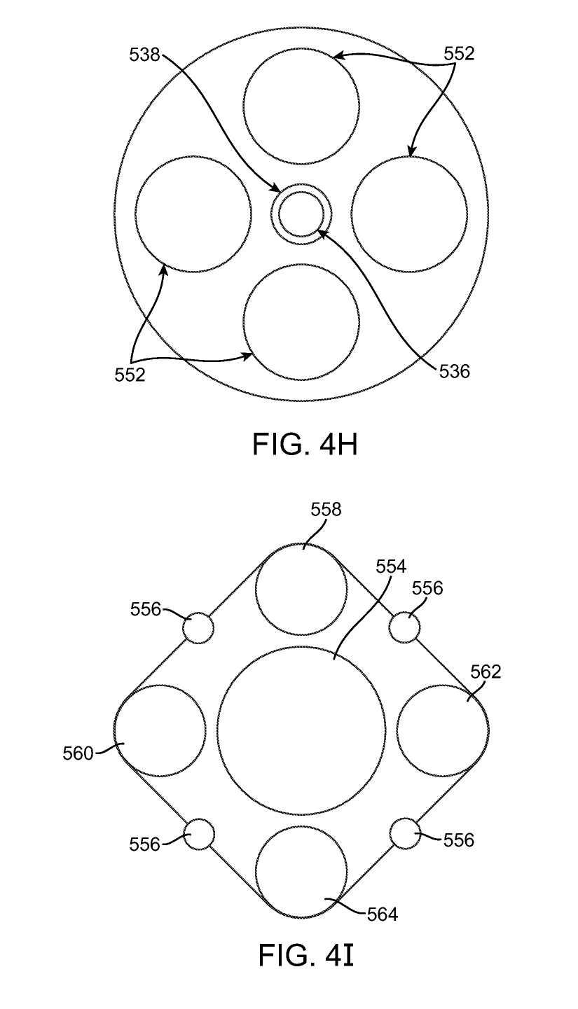

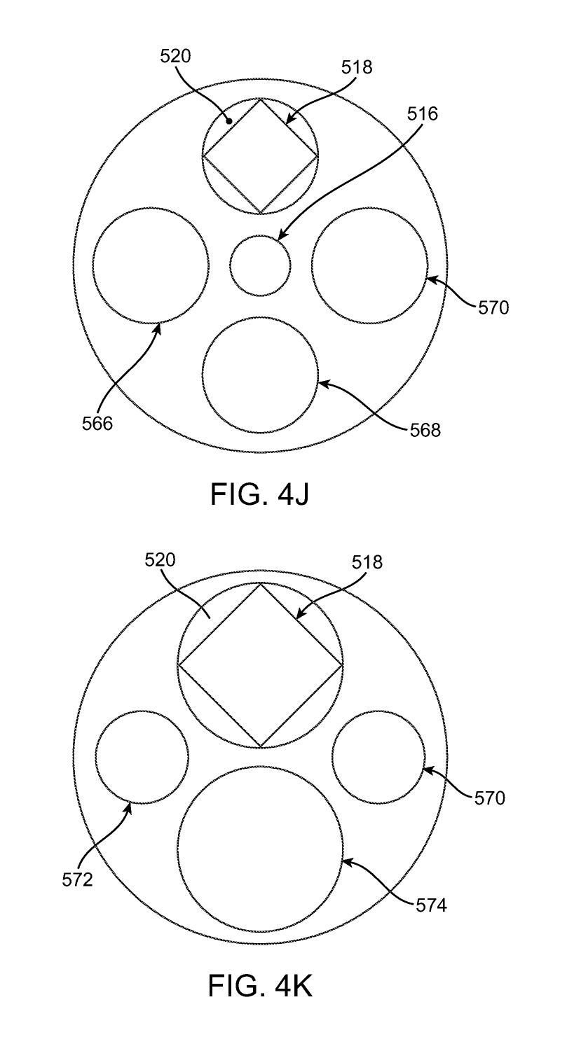

FIGS. 4A through 6 illustrate cross sectional views to depict the relative positioning of various features in the enlongate instruments. FIG. 4A illustrates a partial cross sectional view of a main flexible instrument (320) in which various ports for various lumens in the main flexible instrument are shown in more detail. Insufflation, irrigation, and aspiration may be common procedures in a surgical operation. The various lumens and ports in the main flexible instrument may be used to perform these procedures. In this embodiment, the main instrument may include lumens and associated ports (402, 404, 406, 408, 410, and 412). Lumen and associated port (402) may be used to house a sensory or imaging system (e.g., various types of cameras, other optical or non-optical systems, etc.). Lumen and associated port (404) may be used as a channel to pass tools, additional robotic arm to perform various operations, or aspiration port. Lumens and associated ports (406 and 408) may be used to house various illumination or lighting system. Lumen and associated port (410) may be used to as an irrigation port. Lumen and associated port (412) may be used as an insufflation port.

FIG. 4B illustrates another embodiment of a main or junction instrument (400). In this embodiment, the main instrument (400) includes dedicated fluid flush (500) and fluid egress (506) lumens. These lumens allows for maintained fluid flush and egress by not compromising lumen space with surgical tools. This may be an important aspect to maintain control or to prevent extravasations. The depicted embodiment also comprises an optics bundle (504) surrounded by illumination light fibers (502) and a working channel (508) configured to accommodate movable coupling (i.e., relative roll, relative insertion/retraction) of surgical instruments.

FIG. 4C illustrates another embodiment of a main or junction instrument. In this embodiment, the main instrument cross section includes a dedicated, clearance fit laser channel (516). This separate channel (516), which may be configured to have a diameter close to that of biggest laser fibers available, provides repeatable entrance of the laser into the optical field. The embodiment depicted in FIG. 4C also comprises three working channels (510, 512, 514) to accommodate instruments, fluid flush, fluid egress, and the like, as well as an image capture device chip (518), such as a CMOS or CCD chip in the range of one by one millimeter dimensions, surrounded by four quadrants of light fibers (520).

FIG. 4D depicts an embodiment having an optics bundle (522) surrounded by a light fibers (524), and a relatively large working channel (526) for fluid flush or deployment of instruments, such as safety wires, baskets, laser instruments, or localized medications, such as into or directly at tumors or other tissue structures or lesions. FIG. 4E depicts an embodiment having an optics bundle (528) surrounded on two sides by light fiber bundles (530, 532). This embodiment also facilitates a relatively large working channel (534). FIG. 4F depicts an embodiment having an optics bundle (536) surrounded by light fibers (538), a relatively small working channel (540), which may be utilized, for example, for laser fibers, cautery tools, and fluid egress, and a relatively large working channel (550), which may be utilized, for example, for instrument deployment or fluid flush. FIG. 4G depicts an embodiment having an optics bundle (536) surrounded by light fibers (538), and three working channels (544, 546, 548) which may be variably used for fluid flush, fluid egress, and relatively small surgical tools such as nitinol baskets, needles, laser fibers, and cautery devices. FIG. 4H depicts a 5 lumen configuration having an optics bundle (536) surrounded by concentric light fibers (538) positioned centrally, and four working channels positioned symmetrically to provide certain kinematic homogeneity properties which may be valuable for bending control and the like. The working channels may be utilized, for example, for fluid flush, fluid egress, and relatively small surgical tools such as nitinol baskets, needles, laser fibers, and cautery devices. FIG. 4I depicts another 5 lumen design wherein a relatively large working channel (554) is positioned centrally, and four corner lumens are provided to accommodate a scope (558), laser (562), flush (564), and lighting or surgical instrumentation (560) with a substantially square cross section as depicted. The lumens (558, 560, 562, 564) may be used in various combinations and permutations to accommodate various functionalities and instrumentation, such as fluid flush, fluid egress, and relatively small surgical tools such as nitinol baskets, needles, laser fibers, and cautery devices. Also shown are four pullwire lumen locations (556) positioned to accommodate steering of the cross section with tension members such as pullwires, or by compressive/pushrod type members. FIG. 4J depicts an embodiment wherein a CMOS or CCD chip (518) is surrounded closely by four sectors of light fibers (520), and wherein a central laser channel (516) and three working channels (566, 568, 570) are provided to accommodate fluid flush, fluid egress, and relatively small surgical tools such as nitinol baskets, needles, laser fibers, and cautery devices. The imaging chip (518) preferably has 1 mm by 1 mm dimensions, and the overall diameter of the cross section depicted preferably is about twelve French. The embodiment depicted in FIG. 4K has a similar image capture (518) and lighting (520) configuration with two relatively small working channels and one relatively large working channel. The working channels may be utilized for fluid flush, fluid egress, and relatively small surgical tools such as nitinol baskets, needles, laser fibers, and cautery devices.

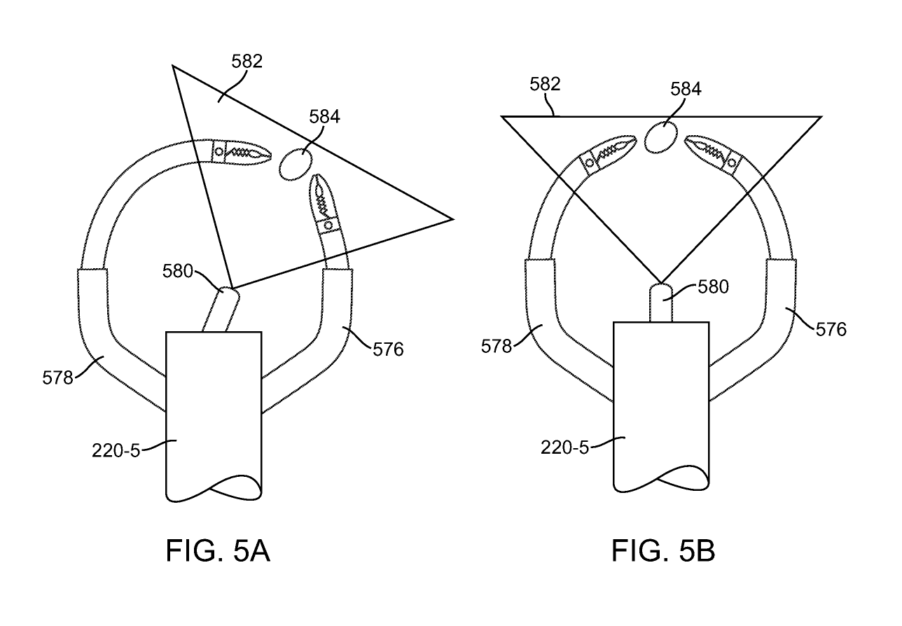

As described above, it may be desirable to have a movable image capture device (580), or at least a movable field of view (582) pertinent to an image capture device, that may be configured and controlled to keep certain objects (584) or portions of instruments (576, 578), such as their distal portions, within the field of view. Such a configuration is depicted in FIG. 5, wherein two instrument assemblies exit from a main or junction sheath instrument (220-5) and address an object such as a tissue structure (584). Similarly control of tools operatively coupled to a setup or junction sheath may be configured such that as the junction sheath moves, the tools may also maintain their position with respect to their target anatomy (584). Various control modes may be utilized to coordinate an endoscope or image capture device (580) operatively coupled to a setup or junction sheath (220-5) and one or more operational instruments, including, for example, engaging an image maintaining mode; allowing for motions with an input device, such as a master input device, buttons on a pendant button console, or voice commands, to select the direction for the setup sheath to move. The movements may be velocity or position controlled, and could be configured, for example, to move the junction sheath in one desired direction while simultaneously moving the endoscope or image capture device. In another embodiment, movements could be coordinated such that the tangent of the endoscope continues to pass through the center of the desired target.

In another embodiment, the controls configuration could assume that a phantom target is some nominal distance (for instance 3 inches) from the endoscope, and move the endoscope to keep that phantom target in view. In another embodiment, the controls configuration could assume the phantom target lies between or near the end effectors or distal portion of one or more instruments, and track such phantom location. Image processing may be utilized to detect the large eigenvalues (features) in the image frame and maintain the majority of those features in the same position in the image. This is like image stabilization where the system is deliberately rejecting the setup or junction sheath motion. In another embodiment, the controls configuration may allow the user to select the desired target via an interface such as a trackball, and track that feature using large eigenvalue tracking or an image swatch correlator. The operator may be able to controllably disengage an image maintaining mode.

In another embodiment, the controls configuration may be set up to keep the tools in the same position in the patient reference frame while moving the setup or junction sheath around. With such configuration, a localization modality may be utilized to understand the position and/or orientation of the main sheath, and/or instruments or portions thereof. Suitable localization modalities or technologies include but are not limited to electromagnetic localization configurations, such as those available from the Biosense division of Johnson & Johnson, Inc under the tradename CartoXP RTM, potential difference based position sensors, such as those available from the Endocardial Solutions division of St. Jude Medical under the tradename EnSite RTM, and fiber bragg based solutions, such as those available from Luna Innovations, Inc., of Roanoke, Va. Open loop kinematic analysis may also be utilized to determine such positions and/or orientations, as described, for example, in some of the aforementioned incorporated references. Also as described in some of the aforementioned incorporated references, a sheath moving model may be selected, the sheath moved utilizing commands from, for example, a master input device, control buttons on a pendant control pad, voice commands, and the like. The control paradigm could be velocity or position control. End effector movement at the distal portion of a given instrument may be conducted by calculating the motion of the end effectors as a function of sheath movement, and moving the end effectors in the equal and opposite direction. Another suitable modality employs serving the end effector position in a global frame during the setup or junction sheath motion.

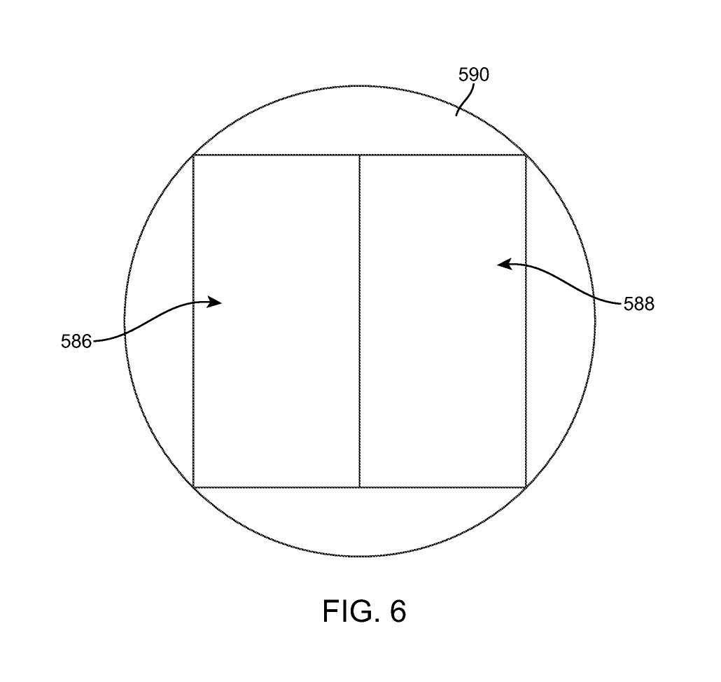

FIG. 6 depicts a split lens design which may be utilized to yield two independent images on the same camera package (590). Referring to FIG. 6, one image (586) can provide low resolution with a large field of view, while another may provide an image with relatively high resolution and a relatively narrow field of view (588). Such configuration may be accomplished utilizing CMOS or CCD technologies, for example, and may be desired in interventional scenarios wherein fine detail is desired for a particular instrument movement or operation, but a broader field of view landscape should be observed as well for safety or simply additional information purposes, at a lower resolution.

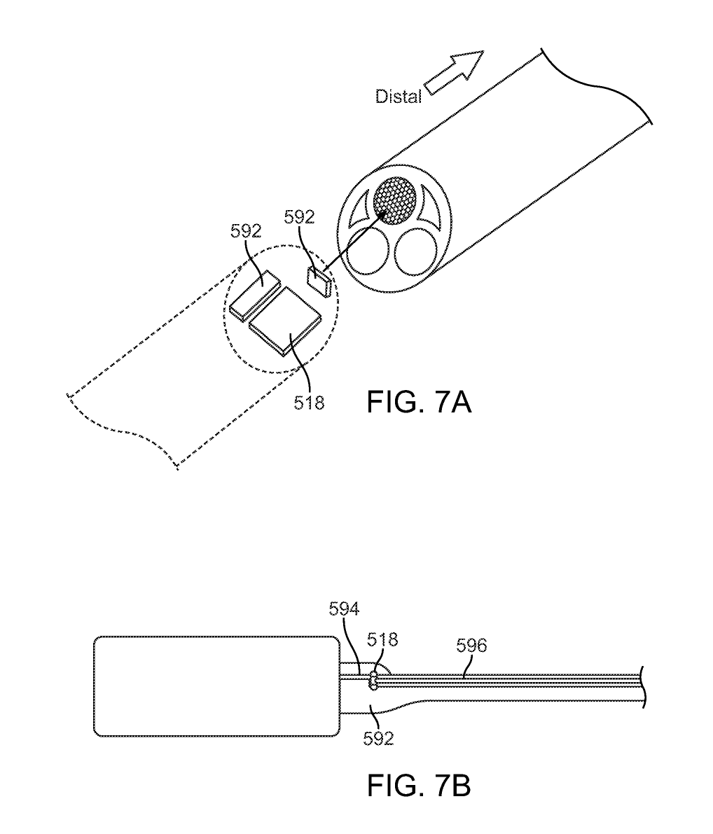

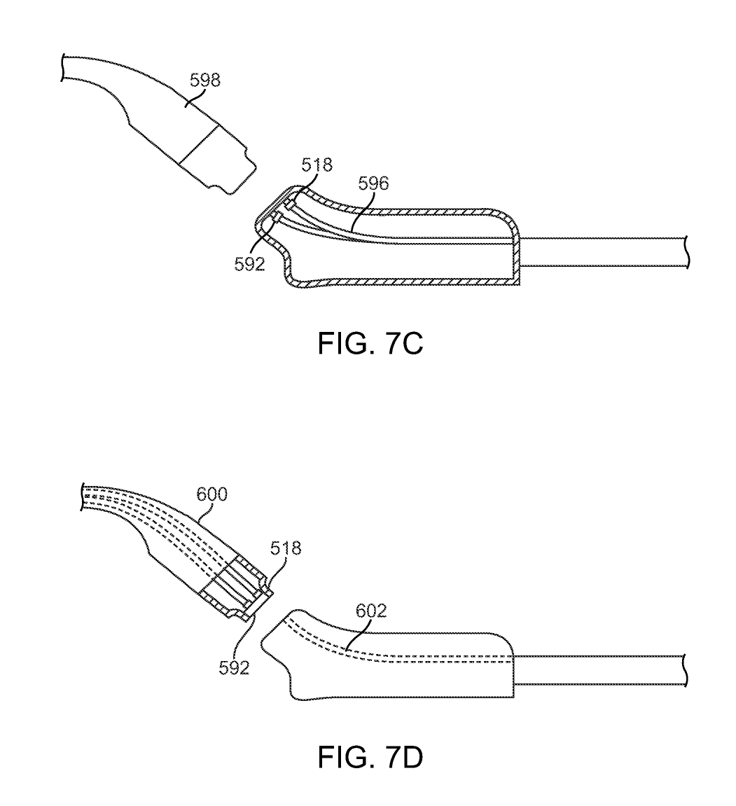

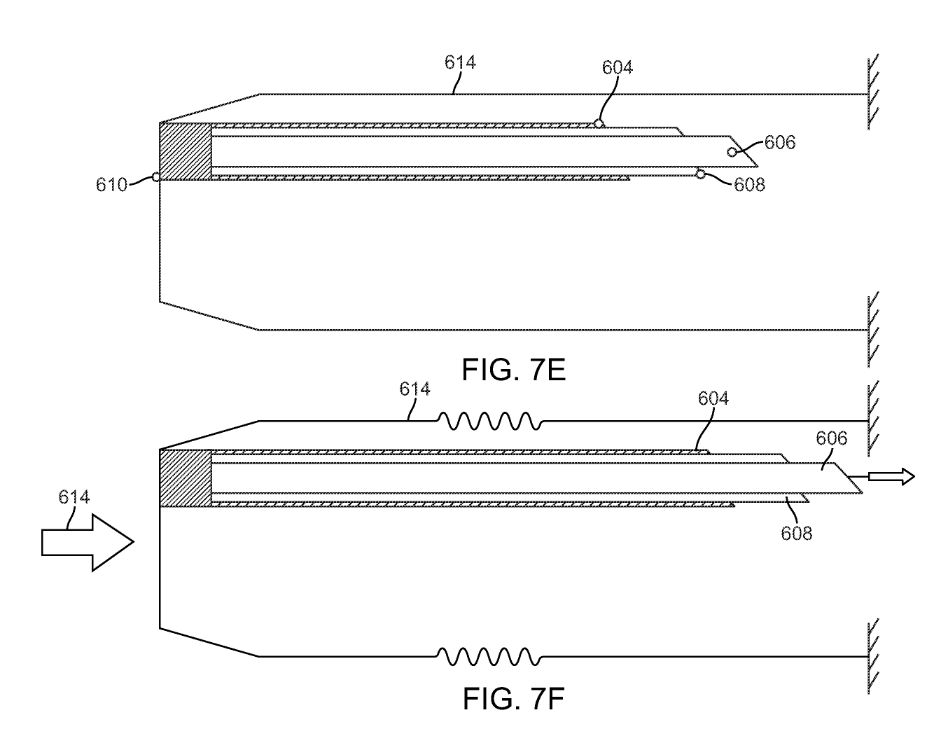

FIGS. 7A-7F depict various aspects of image capture related designs. Referring to FIGS. 7A and 7B, a distal sheath portion comprising an optics bundle and illumination fiber bundles (596) may be interfaced with a proximal portion comprising one or more LEDs (592) for illumination of the illumination fiber bundles (596) and a 1 mm CMOS or CCD imaging chip (518) for image capture from the distal fiber bundle or a rod lens. Wires (594) preferably are utilized to energize the chip (518) and transmit information proximally. The imaging chip and LEDs in this embodiment preferably are positioned proximally of the disposable sheath portion, in one embodiment just distal of a housing to sheath connection. This allows for economic advantages with the disposable aspects, and provides room to position and connect the digital optic components.

FIG. 7C depicts an embodiment wherein a handpiece with a digital interface (598) is removably couplable to a distal portion comprising an imaging chip (518), one or more LEDs (592), and one or more illumination light fiber bundles (596). FIG. 7D depicts an embodiment wherein a handpiece (600) comprises an imaging chip (518) and one or more LEDs (592), and wherein the distal and removably uncouplable portion comprises light/optics fiber bundles (602) to make for a relatively inexpensive disposable distal portion. Referring to FIGS. 7E and 7F, embodiments such as those described above in reference to FIGS. 7A-7D are composites comprising various subportions having various Young's moduli and other material properties. Light and/or optics transmission fibers, for example, generally are not as compressible as the materials which may comprise polymeric sheath instruments. To accommodate relative motion without breakage during loading, all of the portions may be anchored at one end of the assembly, while they may be allowed to “float” relative to each other in other portions of the assembly. As shown in FIGS. 7E and 7F, with a compressive load (612) applied, a polymeric sheath body (614) compresses more than a light fiber bundle (608) or optics bundle (606), and relative motion between these the light fiber structures (606, 6078) and the sheath body (614) is facilitated by an intermediate layer comprising a tubing or applied layer of a material such as polyimide which is selected for its friction and shape maintenance properties. As a result of such a configuration, the sheath body (614) may be compressed while the fiber structures (606, 608) float in isolation by the intermediate layer (604), compress less, if at all, and remain undamaged. Compression of a sheath body may occur in surgical operation, during various setup testing configurations, and during bending of the sheath body where fibers are not at the direct bending center of the structure and therefore undergo some tension or compression.

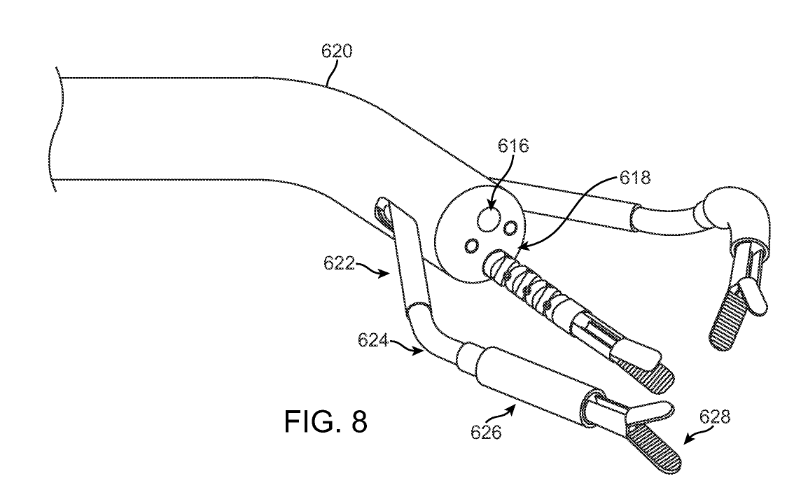

Referring to FIG. 8, a three-armed configuration is depicted having a flexible junction sheath (620), two grasping instruments each comprising a shoulder (622), elbow (624), wrist (626), and grasper (628), an image capture device (616) such as a camera, and an additional tool port (618) to accommodate yet another controllable instrument assembly. Such a configuration may be utilized for improved tissue manipulation and/or therapy while tissue is being manipulated. The arms preferably have pitch and yaw capabilities in the wrist and elbow segments, which are coupled in tandem. The shoulder also preferably has pitch and yaw capability, and also roll. The elbow and wrist are axially coupled and may be inserted and withdrawn from the inside of the shoulder. Preferably they are also geometrically keyed with the shoulder to prevent relative rotation. The entire arm is configured to rotate or roll when the shoulder is rotated. The tool port allows for a third instrument assembly or tool to be introduced; it may be a third arm, cautery tools, a camera tool, therapy delivery device, or other operative device.

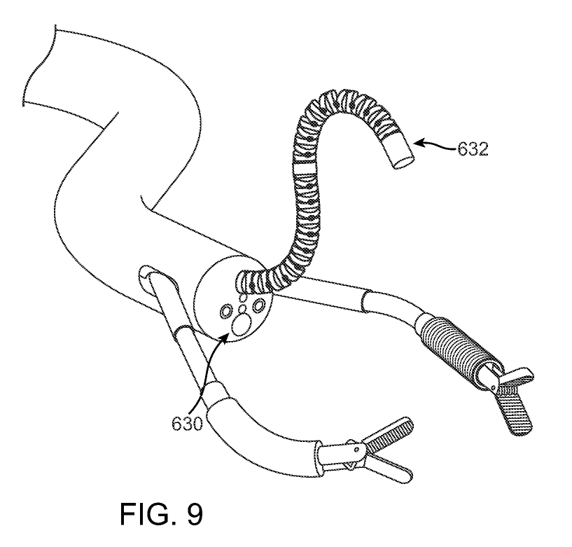

FIG. 9 depicts an embodiment wherein an image capture device (532) such as a camera is mounted upon a controllably steerable, flexible tool to allow the operator to maneuver the entire assembly and tools/instruments without impacting the position of the image from the image capture device. The flexible image capture assembly may be configured to automatically move in order to compensate for motion at its base. A series of two image capture devices may be used during a procedure—a main image capture device through a tool or image capture port (630) on the junction sheath, and a second image capture device (632) on a flexible construct as shown. With such a configuration, the operator has two available images, each of which may have different properties, such as field of view, focal length, and resolution.

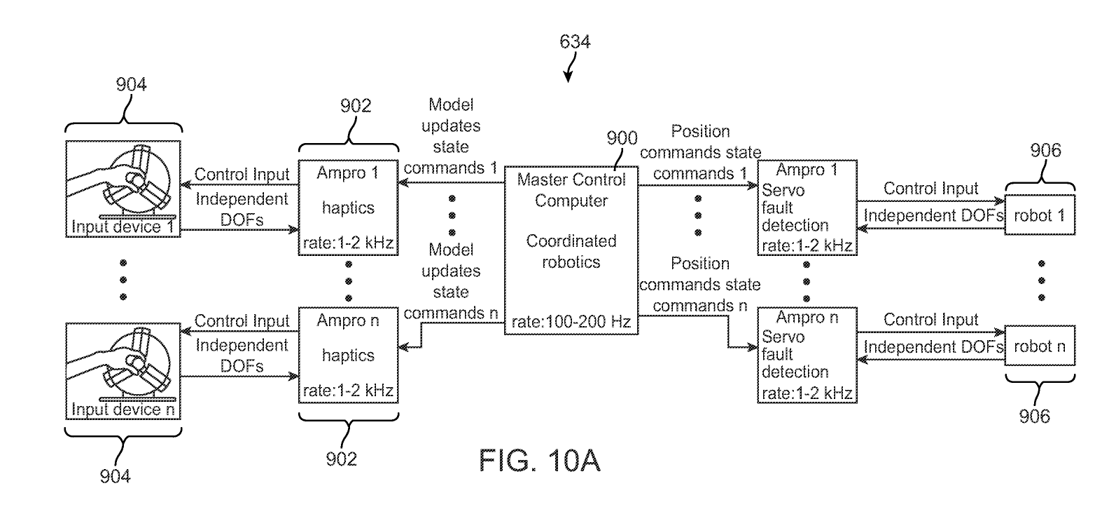

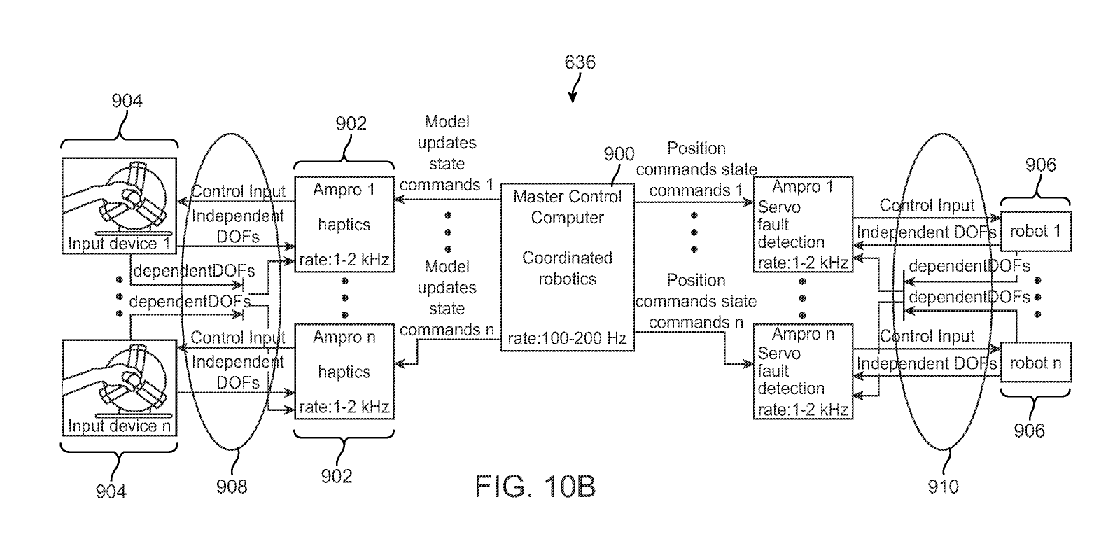

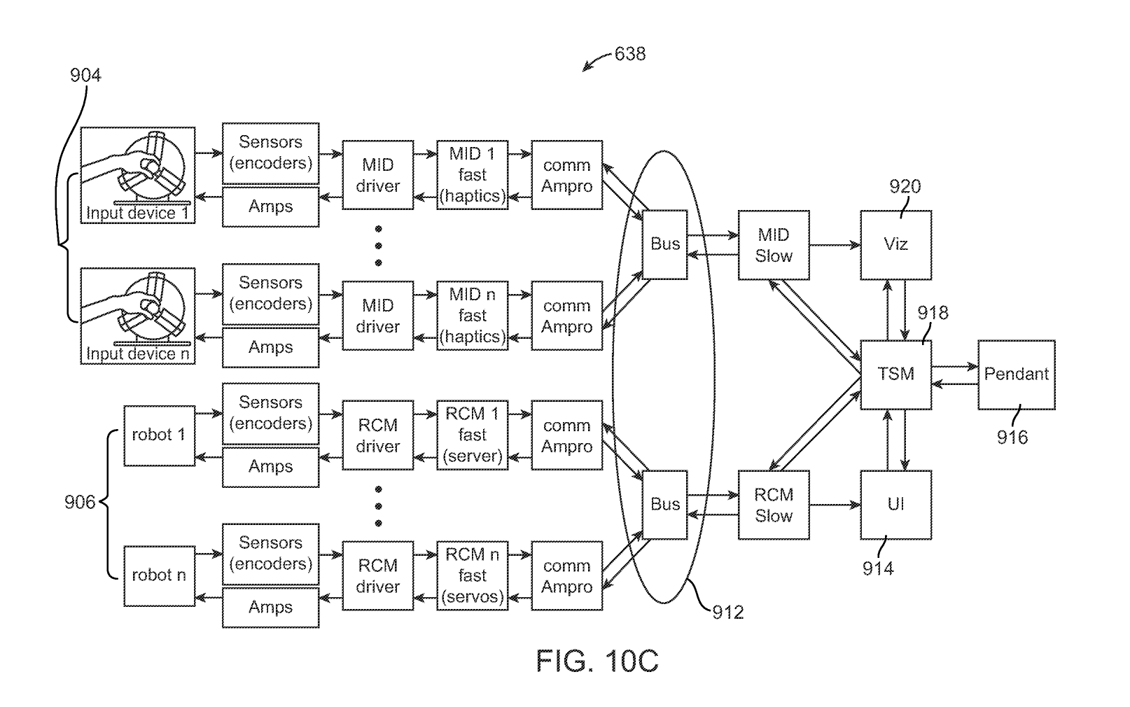

Referring to FIGS. 10A-10C, various scaleable control architecture aspects are illustrated to address systems such as that depicted in FIG. 2A, which may be described as multi-robot systems requiring controls coordination. In an environment wherein computational power is infinite, scaling challenges are more easily addressed. In the operating room, scaling is required, and unlimited computational resources are not present. One of the challenges is that surgical motions, such as the motions of a surgeon's hands, occur on the order of 1-5 Hz. To represent them well and capture the intent of the surgeon or operator, these motions may be sampled at an input device at frequencies such as 100-200 Hz. Servo systems run stably at frequencies between 1 and 3 KHz; motors require higher bandwidth for the kind of frequency dynamics and response we desire from robotic systems for the surgical environment. One of the ways to scale computationally is to break up the trajectory planning for surgical motions and compute with a powerful main computer, or “Master Controller Computer” (900) to organize all of the degrees of freedom and run then at 100-200 Hz, as in the architecture (634) diagrammed in FIG. 10A. Then a series of other computers (902) may each take on a discrete group of degrees of freedom, say 10 or so degrees of freedom per computer, and close the controls loop at 1-3 KHz. Thus, their commands come in at 100-200 Hz, and they servo/resolve those commands at 1-3 HHz. In the embodiment (634) of FIG. 10A, the degrees of freedom are discretely packaged by design—on the input side (904) and on the robots side (906). Referring to FIG. 10B, another embodiment (636) is depicted, which is similar to that depicted in FIG. 10A with the exception that a system of busses (908, 910) distributes all of the information to all of the input devices (904) and robots (906). Thus, this architecture is more flexible because all of the information regarding all of the degrees of freedom is cross coupled and may be managed and processed systematically. Referring to FIG. 10C, another architecture embodiment (638) is depicted incorporating the bus system architecture of the embodiment of FIG. 10B and distributing the computation to control multiple robots with multiple computation structures, all attached to one major bus (912). This configuration also incorporates unified state behavior (for example, during states of setup configuration, master following, detected errors, etc) with the state machine (918) and computing related to the user interface (914), pendant control button control interface (916), graphical visualization system (920) all functioning together in a unified way at a slower bandwidth.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A minimally invasive robotic interventional system, comprising:
a. a first instrument driver operatively coupled to a first flexible instrument assembly;
b. a second instrument driver operatively coupled to a second flexible instrument assembly; and
c. an elongate junction sheath having a proximal end, a distal end, and a body portion therebetween, wherein the distal end is coupled to an image capture device and configured to be positioned inside a patient's body adjacent one or more targeted tissue structures, the proximal portion is configured to be positioned external to an opening in the patient's body, and the body portion is configured to contact an entry point of the junction sheath into the patient's body;
wherein a distal portion of the of the first flexible instrument assembly is movably coupled through a first lumen defined through the junction sheath between the proximal and distal ends of the junction sheath, wherein a distal portion of the second flexible instrument assembly is movably coupled through a second lumen defined through the junction sheath between the proximal and distal ends of the junction sheath, and wherein the image capture device is positioned substantially equidistantly in between the first and second lumens of the junction sheath to have a field of view configured to capture activity of each of the flexible instrument assembly distal portions.

2. The system of claim 1, wherein the image capture device comprises a rod lens.

3. The system of claim 1, wherein the image capture device comprises a chip selected from the group consisting of a CMOS imaging chip and a CCD imaging chip.

4. The system of claim 1, wherein the image capture device is movable relative to the elongate junction sheath.

5. The system of claim 4, wherein the image capture device may be teleoperably moved relative to the elongate junction sheath following commands input by an operator to an operator workstation.

6. The system of claim 5, wherein the image capture device may be teleoperably moved utilizing an actuation that is electromechanical or manual.

7. The system of claim 4, wherein the image capture device may be reoriented relative to the elongate junction sheath.

8. The system of claim 4, wherein the image capture device may be inserted or retracted relative to the elongate junction sheath.

9. The system of claim 1, wherein at least one of the first and second lumens of the elongate junction sheath comprises an arcuate lumen configured to cause an instrument coupled therethrough to diverge away from an instrument coupled through the other of the first and second lumens as said instruments exit the distal portion of the elongate junction sheath.

10. The system of claim 9, wherein both the first and second lumens of the elongate junction sheath comprise arcuate lumens configured to cause the first and second instruments to diverge away from each other as they exit the distal portion of the elongate junction sheath.

11. The system of claim 10, wherein the arcuate lumens are configured to cause the first and second instruments to converge toward each other as they enter the proximal portion of the elongate junction sheath.

12. The system of claim 1, wherein the first instrument assembly comprises an outer sheath instrument coaxially coupled to an inner guide instrument, which is coaxially coupled to a working instrument, and wherein the first instrument driver is configured to independently and simultaneously provide multi-degree-of-freedom actuation to each of the outer sheath instrument, inner guide instrument, and working instrument, in accordance with commands issued by an operator utilizing an operator workstation having a user interface.

13. The system of claim 12, wherein the user interface comprises one or more master input devices configured to intake three dimensional positional navigation instructions from the operator pertinent to positional control of at least one point upon one of the instruments comprising the assembly.

14. The system of claim 13, wherein two or more master input devices are utilized to intake three dimensional positional navigation instructions from the operator pertinent to positional control of at least one point upon each of at least two of the instruments comprising the assembly.

15. The system of claim 12, wherein each of the outer sheath instrument, inner guide instrument, and working instrument is remotely controllable in at least six degrees of freedom.

16. The system of claim 15, wherein the first instrument driver is configured to pitch, yaw, and insert each of the outer sheath instrument, inner guide instrument, and working instrument independently and simultaneously.

17. The system of claim 16, wherein the first instrument driver is configured to roll each of the outer sheath instrument, inner guide instrument, and working instrument simultaneously and together.

18. The system of claim 17, further comprising a controller configured to automatically reorient the distal portion of the working instrument as the instrument assembly is rolled, to retain positioning of the distal portion of the working instrument while it is being reoriented.

19. The system of claim 12, wherein second instrument assembly comprises an outer sheath instrument coaxially coupled to an inner guide instrument, which is coaxially coupled to a working instrument, and wherein the first instrument driver is configured to independently and simultaneously provide multi-degree-of-freedom actuation to each of the outer sheath instrument, inner guide instrument, and working instrument, in accordance with commands issued by an operator utilizing an operator workstation having a user interface.

20. The system of claim 19, further comprising a controller configured to coordinate the motion of each of the first and second instrument assemblies relative to each other and relative to the field of view of the image capture device as the instrument assemblies are remotely actuated by independent instrument drivers in accordance with commands from the operator workstation.
